(19) United States
(12) Patent Application Publication
(10) Pub. No.: US 2015/0374892 A1
(43) Pub. Date: Dec. 31, 2015

(54) CABLE SYSTEM FOR IMPLANTABLE BLOOD PUMP WITH ACCIDENTAL DISCONNECTION PREVENTION

(71) Applicant: THORATEC CORPORATION, Pleasanton, CA (US)

(72) Inventors: Masamichi YANAI, Ann Arbor, MI (US); Diane L. THOMPSON, Belleville, MI (US); Himanshu K. BHATT, Ann Arbor, MI (US)

(21) Appl. No.: 14/761,092

(22) PCT Filed: Jan. 22, 2014
(86) PCT No.: PCT/US2014/012502

§ 371 Date: Jul. 15, 2015

Related U.S. Application Data
(60) Provisional application No. 61/755,497, filed on Jan. 23, 2013.

(51) Int. Cl. A61M 1/10; A61M 39/10; H01B 9/00; A61M 1/12

(52) U.S. Cl. CPC A61M 1/1008; A61M 1/127; A61M 39/10; H01B 9/00; A61M 2039/1022

(57) ABSTRACT

A blood pump system includes a pump that is to be implanted in a patient and a control unit disposed externally of the patient. A percutaneous cable section is configured to pass percutaneously through an incision in the patient and has first and second ends. The second end is connectable to the pump. An extension cable section has second ends, and the second end is connectable to the control unit. The first and of the cable sections removably mate together and include a push-pull electrical connector covered by a secondary threaded mechanical connector.

### BACKGROUND OF THE INVENTION

The present invention relates in general to circulatory assist devices, and, more specifically, to enhanced reliability and prevention of inadvertent disconnection of a percutaneous cable connection.

Many types of circulatoty assist devices are available for either short term or long term support for patients having cardiovascular disease. For example a heart pump system known as a left ventricular assist device (LVAD) can provide long term patient support with an implantable pump associated with an externally-worn pump control unit and batteries. The LVAD improves circulation throughout the body by assisting the left side of the heart in pumping blood. One such system is the DuraHeart® LVAS system made by Terumo Heart, Inc., of Ann Arbor, Mich. The DuraHeart® system employs a centrifugal pump with a magnetically levitated impeller to pump blood from the left ventricle to the aorta. An electric motor magnetically coupled to the impeller is driven at a speed appropriate to obtain the desired blood flow through the pump.

A typical cardiac assist system includes a pumping unit, electrical motor (e.g., a brushless DC motor integrated in the pump housing), drive electronics, microprocessor control unit, and an energy source such as rechargeable batteries and/or an AC power conditioning circuit. The system is implanted during a surgical procedure in which a centrifugal pump is placed in the patient's chest. An inflow conduit is pierced into the left ventricle to supply blood to the pump. One end of an outflow conduit is mechanically fitted to the pump outlet and the other end is surgically attached to the patient's aorta by anastomosis. A percutaneous cable connects to the pump, exits the patient through an incision, and connects to the external control unit. For practical reasons, it is preferable that the percutaneous cable extends for only a short distance from the incision. A cable connector is provided at the end of the percutaneous cable in order to connect with an extension cable coming from the external controller.

In the event, of any problems or failure of the external control unit, it may become necessary to replace it. Therefore, a removable connection is provided for the percutaneous cable. The electrical and mechanical interconnection functions of the inline connector are critical to the patient. It must be secure and not subject to accidental disconnection. On the other hand, if the control unit needs to be replaced due to a failure or potential failure then it should be quick and easy to disconnect and then reconnect the inline connector. Thus, it would be desirable to provide a connector that simultaneously meets the contradictory requirements of being secure and easy.

### SUMMARY OF THE INVENTION

The connector of the invention is both secure and easy as a result of combining two easy locking mechanisms. A primary connection is realized by an electrical push-pull locking connector which is covered by a secondary threaded (rotatable) mechanical connection. The secondary locking mechanism protects the primary locking mechanism while preventing accidental disconnection of the easy to remove push-pull connector. The body of the secondary connector may have grip features that reduce slipping and give an indication to the user that it can be disconnected by rotation. The secondary connector also acts as a cable strain relief feature to lessen the chance of wire fracture at the connector.

### BRIEF DESCRIPTION OF THE DRAWINGS

FIG. 1 is a diagram of a circulatory assist system as one example of an implantable pump employing the present invention.

FIG. 2 is a block diagram of a circulatory assist system with an inline connection between an implanted pump and a control unit.

FIG. 3 is a plan view of a percutaneous cable according to one embodiment of the present invention.

FIG. 4 is an end view of a connector element of FIG. 3.

FIG. 5 is a cross-sectional view of the cable of FIG. 3.

FIG. 6 is a cross-sectional view of the connector element of FIG. 5.

FIG. 7 is a cross-section view taken along line 7-7 of FIG. 6.

FIG. 8 is a perspective view of an extension cable with a connector element that connects to the percutaneous cable shown with a secondary locking element in a retracted position.

FIG. 9 is a plan view of the connector element of FIG. 8 shown with the secondary locking element in an extended position.

FIG. 10 is a cross-sectional view of the connector element of FIG. 9.

FIG. 11 is a cross-sectional view of the secondary locking element.

### DETAILED DESCRIPTION OF PREFERRED EMBODIMENTS

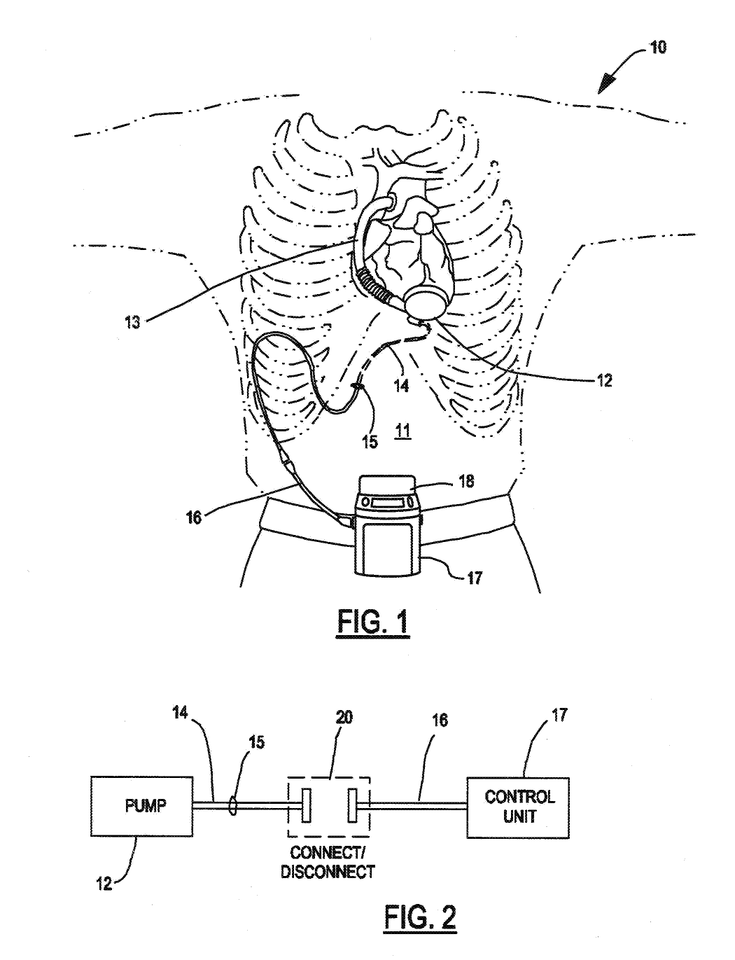

Referring to FIG. 1, a patient 10 is shown in fragmentary front elevational view. Surgically implanted either into the patient's abdominal cavity or pericardium 11 is the pumping unit 12 of a vermicular assist device. An inflow conduit (on the hidden side of unit 12) pierces the heart to convey blood from the patient's left ventricle into pumping unit 12. An outflow conduit 13 conveys blood from pumping unit 12 to the patient's aorta. A percutaneous power/communication cable 14 extends from pumping unit 12 outwardly of the patient's body via an incision 15 to an extension cable 16 which further connects to a control unit 17 worn by patient 10. Control unit 17 is powered by a main battery pack 18 and/or an external AC power supply and an internal backup battery.

As shown in FIG. 2, a removable connector 20 joins cables 14 and 16 in a manner that allows disconnection and reconnection when desired. Due to its critical nature, this connection must be secure. However, a quick and easy disconnection/re-connection is also needed since any replacement of control unit 17 must be done without complications.

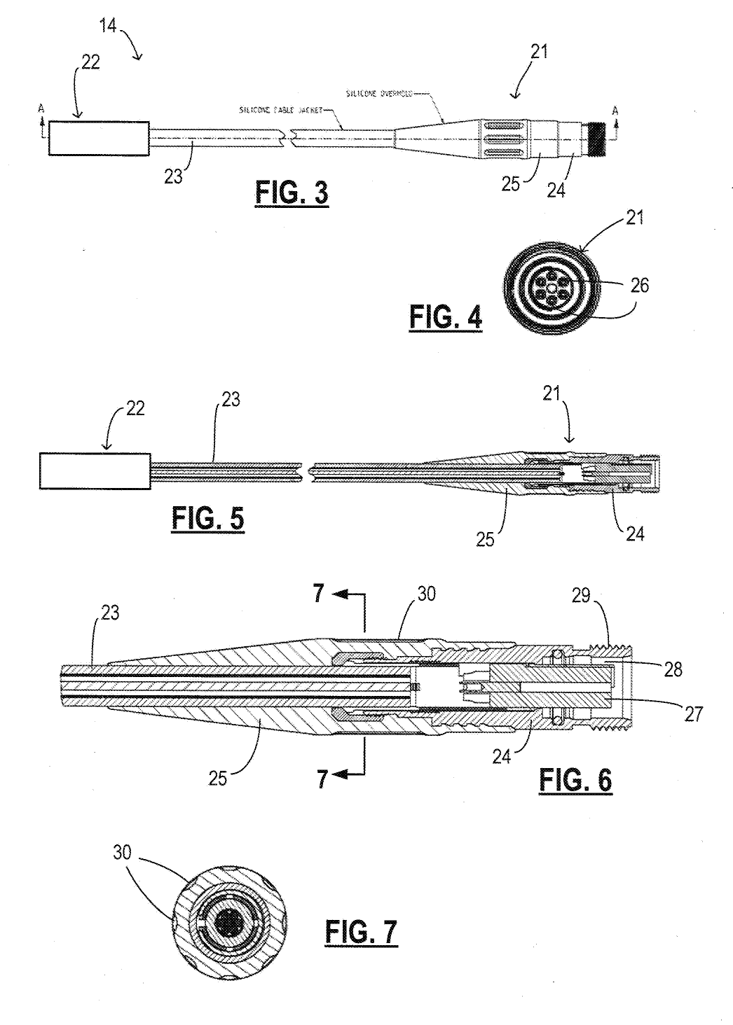

Percutaneous cable 14 is shown in greater detail in FIGS. 3-7. it has a first connector element 21 at one end for mating with the extension cable and has a connector element 22 at the second end for mating with the pump unit. A central cable run 23 extends between connector elements 21 and 22 and may preferably be covered with a silicone cable jacket. As shown in FIG. 3, an alter surface of connector element 21 includes a first body segment 24 and a second body segment 25. FIG. 4 is an end view showing a plurality of cable conductors 26 for carrying electrical signals for driving the pump unit.

As shown in FIGS. 5 and 6, connector element 21 includes a push-pull primary electrical connector element 27 fixedly mounted within a cylindrical bore of body segment 24. Connector element 27 may be comprised of a push-pull Fischer connector (available from Fischer Connectors S.A., Alpharetta, Ga.) or a push-pull Lemo connector (available from LEMO S.P.A. Emblem, Switzerland). Other types of latching or locking connectors can also be used such as a BNC connector. Alternatively, a non-locking, connector could be used since the secondary mechanical connector of the invention would keep the primary electrical connector securely connected.

Annular open space 28 is provided around connector element 27 in order to receive the other portion of the push-pull connector mounted to the extension cable as explained below. Body segment 24 includes a threaded extension 29. Body segment 24 is a metal outer shell of the push-pull connector. A second body segment 25 is over-molded onto body segment 24 and an adjacent portion of cable run 23. Preferably, body segment 25 is formed of a flexible silicone which provides a liquid seal around the cable end connector. A plurality of gripping slots 30 are provided around the periphery of over-molded body segment 25 so that one hand of a user can maintain a grip on connector element 21 while threading or unthreading a mating element of the extension cable as described below.

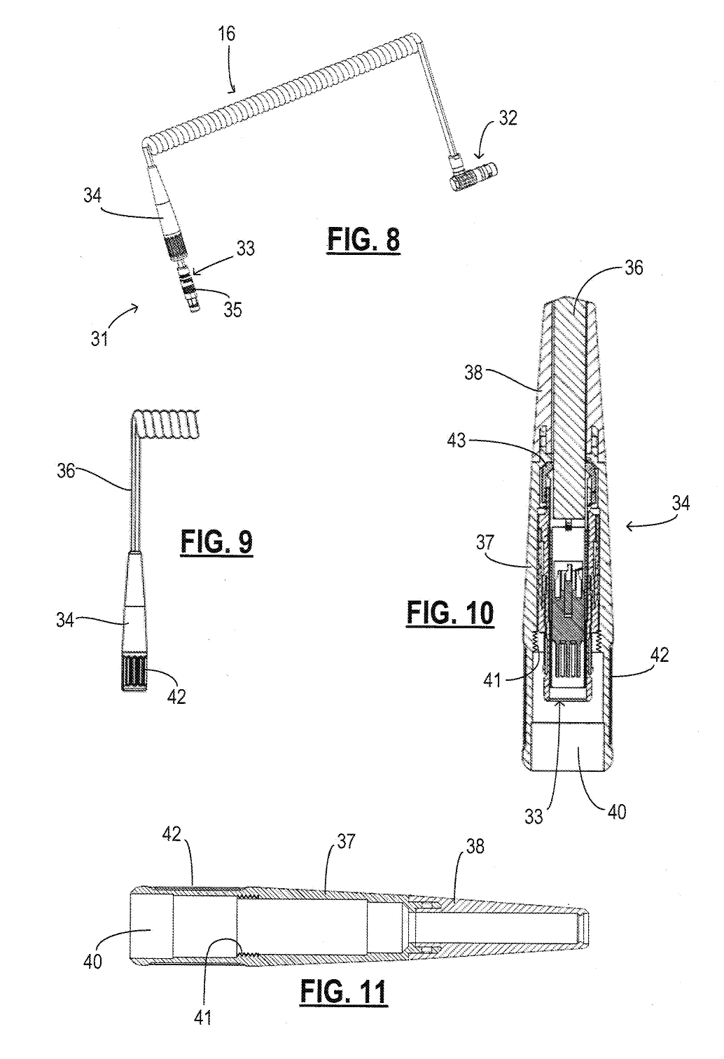

FIG. 8 shows extension cable 16 having a first end 31 for mating with the percutaneous cable and a second end 32 for mating with the control unit. First end 31 includes a push-pull electrical connector element 33 for mating with connector element 27 of the percutaneous cable. A secondary connector element 34 is slidable on extension cable 16 and has a retracted position shown in FIG. 8 for exposing connector element 33. Connector element 33 has a slidable retainer sleeve 35 that can be pulled away from the connection whenever accessibly exposed by secondary element 34 to allow disconnection of the electrical connector. FIG. 9 shows secondary connector element 34 slid downward over connector element 33 along a portion 36 of extension cable 16 in order to cover the electrical connector and to facilitate threading together with body portion 24 (FIG. 6).

FIG. 10 is a cross-section showing secondary connector element 34 in its extended position over electrical connector element 33. Element 34 has a first body portion 37 preferably formed of a hard thermoplastic. Connector element 34 has a second body portion 38 molded onto portion 37 and preferably comprising a soft, flexible silicone overmold. Body portion 37 has a receptacle area 40 for receiving connector element 21 of percutaneous cable 14, whereby the push-pull connection can be made for the electrical connector. Body portion 37 includes internal threads 41 for mating with threads 29 to make a secondary mechanical interconnection. Electrical connector element 33 and cable 36 fit loosely enough within secondary connector element 34 to permit element 34 to rotate thereon. A mechanical stop of the secondary thread connection is implemented by a contact of locking sleeve 37 against a nut 43 of connector. When secondary connection is engaged, it backs up the primary connection between 33 and 27 when cable 36 is being pulled accidently. Thus, the connection of the present invention employs two easily made interconnects that operate in tandem to provide a secure, reliable connection which is easily undone when intended.

What is claimed is:

1. A blood pump system comprising:
a pump configured for implantation in a patient;
a control unit disposed externally of the patient;
a percutaneous cable section configured to pass percutaneously through an incision in the patient. having first and second ends, wherein the second end is connectable to the pump; and
an extension cable section having first and second ends, wherein the second o end in connectable to the control unit, wherein the first ends of the cable sections removably mate together;
wherein the first ends comprise a push-pull electrical connector covered by a secondary threaded mechanical connector.
